(19) United States
(12) Patent Application Publication
(10) Pub. No.: US 2021/0220632 A1
(43) Pub. Date: Jul. 22, 2021

(54) NON-LASER TATTOO REMOVAL METHOD

(71) Applicant: Estetique International LLC, Boca Raton, FL (US)

(72) Inventors: Candice Piroli, Coraopolis, PA (US); Seifalla Sidky, Delray Beach, FL (US)

(73) Assignee: Estetique International LLC, Boca Raton, FL (US)

(21) Appl. No.: 17/154,375

(22) Filed: Jan. 21, 2021

Related U.S. Application Data
(60) Provisional application No. 62/964,089, filed on Jan. 21, 2020.

(51) Int. Cl. A61M 37/00; A61F 13/40; A61K 33/08; A61K 36/886; A61K 31/728

(52) U.S. Cl. CPC A61M 37/0076; A61M 35/006; A61K 33/08; A61M 2210/04; A61K 31/728; A61M 2037/0007; A61K 36/886

(57) ABSTRACT

A non-laser tattoo removal method that includes the steps of cleaning and drying a target area on a user's skin, the target area including at least one tattoo disposed thereon; administering a deracination serum to the target area with a handheld automatic needle assembly having a plurality of needles after the cleaning and drying of the target area; wiping the target area with an absorbent material to remove an extracted pigmented material at least partially composing the tattoo; applying a malleable clay formulation material to cover the target area and leaving the malleable clay formulation material on the target area until forming a dried-on clay formulation; removing the dried-on clay formulation from the target area; and applying a liquid healing agent to the target area after removing the dried-on clay formulation from the target area and covering the liquid healing agent with at least one transdermal drug delivery sheet for selective removal by the user after a healing time period.

### FIELD OF THE INVENTION

The present invention relates to a non-laser tattoo removal method and, more particularly, relates to a non-laser tattoo removal method utilizing a deracination serum, a malleable clay formulation material, a liquid healing agent, and at least one transdermal drug delivery sheet applied to a target area of a user's skin, the target area including at least one tattoo disposed thereon.

### BACKGROUND OF THE INVENTION

Although tattoos and other permanent make-up applications have traditionally been designed to be permanent, a growing consumer demand for removal of these pigmented skin alterations has given rise to the formulation and proliferation of tattoo removal services and methods. Typically, the tattoo removal process entails the use of laser technologies and devices that heat up the sub-dermal pigment, break it into smaller particles, and then drive the pigmented particles deeper into the person's body through the body's white blood cells. This process is not only painful for the individual because the laser scorches the pigmented tattoo particles to break them down, but it is also time-consuming for the user because existing laser tattoo removal methods require numerous treatments spaced out over relatively even increments of time. Furthermore, there is also a possibility that the sub-dermal pigments broken down by lasers cause a negative health effect on the individual. Overall, this costly, time-consuming, painful, and potentially harmful process is one of the few effective treatments available to users looking to permanently remove their body tattoos, permanent make-up, or scalp micro-pigmentation.

Therefore, a need exists to overcome the problems with the prior art as discussed above.

### SUMMARY OF THE INVENTION

In an effort to revolutionize existing permanent tattoo removal methods and provide consumers with a safer alternative to laser-based tattoo removal methods, the present invention embodies a topical and non-laser tattoo removal system and method. Because this tattoo removal method does not comprise, use, or otherwise require any laser devices or technologies, the tattoo removal process is significantly less costly and less painful for consumers. Specifically, the absence of laser technologies means that the target area is not scorched using a high-temperature laser. The present invention comprises a method of treatment steps that includes applying a series of topical compositions to the skin in a series of designated applications, which serve to break up the tattoo color pigments on the target area without releasing the tattoo color pigments into the user's body or blood stream. Ink residue and toxins are lifted up, extracted, and removed from the skin using the topical compositions in the non-laser tattoo removal method. In this way, the tattoo removal process results in a significantly lower toxic impact to the user's body. This less expensive non-laser tattoo removal method gives consumers a viable alternative to laser tattoo removal that features an array of benefits. Specially trained professionals administer at least the first topical ointment in the tattoo removal method to the skin, ensuring proper and effective application.

With the foregoing and other objects in view, there is provided, in accordance with the invention, a non-laser topical tattoo removal method comprising a sequence of consecutive steps for administering and applying a variety of topical compositions, or formulations, to effectively remove body tattoos, permanent make-up, and scalp micro-pigmentation.

In the current embodiment of this invention, the topical system comprises an initial Step of preparing the target area on the client's skin by cleaning and wiping it dry, wherein the target area includes at least one body tattoo, permanent make-up, or scalp micro-pigmentation disposed thereon.

Another Step in the present invention entails using a tattoo machine or other comparable instrument or device that comprises a plurality of needles for substantially the same purpose, to selectively administer a serum formulation to the target area, wherein the serum formulation may comprise a combination of natural ingredients or constituents which loosen the tattoo color pigments embedded in the skin and allow the loosened tattoo color pigments to rise to the surface of the epidermis rather than being driven deeper into the epidermis or into the user's bloodstream.

A further Step comprises wiping the target area clean and dry after administering the serum formulation to the target area.

In accordance with a next Step, a malleable clay formulation material is applied to the target area after the target area is wiped clean and dry, wherein the method of application is not limited to the utilization of any one specific tool, instrument, or method, and the malleable clay formulation material comprises a combination of natural ingredients or constituents which lift or extract the loosened tattoo color pigments to the surface of the epidermis from the deeper levels of the skin.

A next Step requires leaving the malleable clay formulation material on the target area until a dried-on clay formulation is formed.

Another Step comprises cleaning and drying the target area after the dried-on clay formulation is formed to facilitate execution of subsequent Steps.

A final Step entails applying a liquid healing agent to the target area after removing the dried-on clay formulation from the target area and covering the liquid healing agent with at least one transdermal drug delivery sheet for selective removal by the user after a healing time period. This Step may be repeated at subsequent intervals of time, as needed to facilitate the natural healing process of the target area.

Although the invention is illustrated and described herein as embodied in a non-laser topical tattoo removal method, it is, nevertheless, not intended to be limited to the details shown because various modifications and changes may be made therein without departing from the spirit of the invention and within the scope and range of equivalents of the claims. For example, some steps may be combined or consolidated to achieve the same intended function or purpose in fewer steps. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the device. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a process flow diagram depicting a non-laser tattoo removal method for removing body tattoos, permanent make-up, or scalp micro-pigmentation, in accordance with the present invention;

FIG. 2 is a block diagram of an initial first Step in practicing the non-laser tattoo removal method, in accordance with the present invention;

FIG. 3 is a block diagram of a subsequent Step in practicing the non-laser tattoo removal method, wherein a serum formulation is applied to the target area on a client's skin;

FIG. 4 is a block diagram of a further Step in practicing the non-laser tattoo removal method, in accordance with the present invention;

FIG. 5 is a block diagram of a next Step in practicing the non-laser tattoo removal method, wherein a malleable clay formulation material is applied to the target area;

FIG. 6 is a block diagram of a further Step in practicing the non-laser tattoo removal method, in accordance with the present invention;

FIG. 7 is a block diagram of a final Step in practicing the non-laser tattoo removal method, wherein a liquid healing agent and at least one transdermal drug delivery sheet is selectively applied to the target area initially and at selective intervals of time; and

FIG. 8 is a schematic diagram depicting constituents that make up the non-laser tattoo removal method, in accordance with a preferred embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel non-laser tattoo removal method 100 (hereinafter, “the removal method 100”) for the removal of body tattoos, permanent make-up, and scalp micro-pigmentation (wherein body tattoos, permanent make-up, and scalp micro-pigmentation shall be collectively referred to herein as “tattoos” for brevity). Embodiments of the invention provide for several topical constituents or formulations, to be administered or applied to a target area on the skin in a pre-determined sequence, wherein the target area includes at least one tattoo disposed thereon. Practicing the removal method 100 thereby facilitates the effective but non-laser removal of various pigments embedded in a user's epidermis. The removal method 100 may further be effective in removing various other forms and types of temporary or permanent ink or pigmentation not explicitly referenced herein.

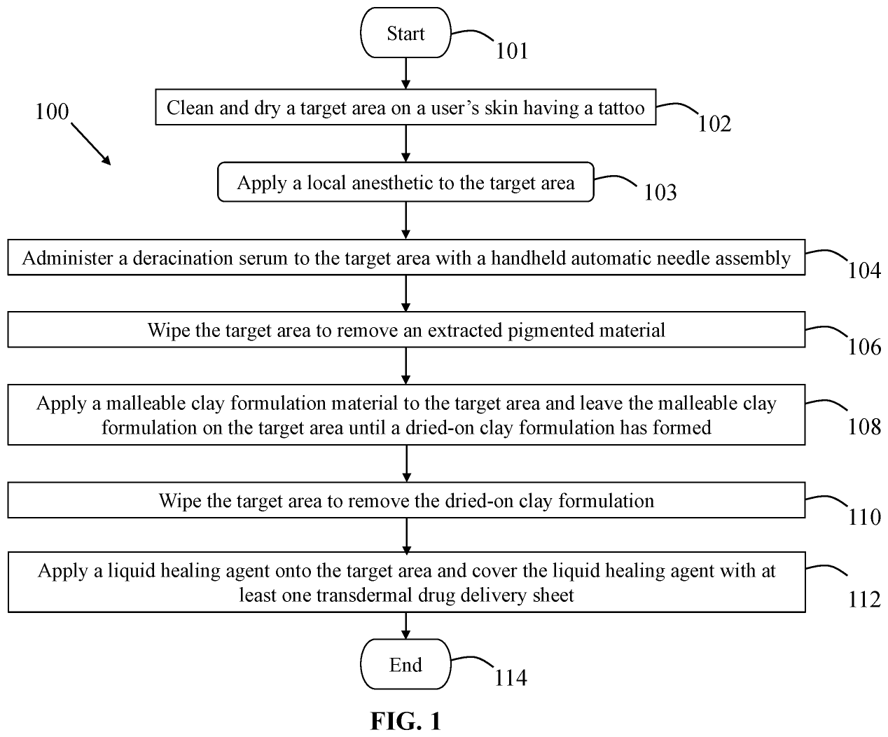

As referenced in the process flow diagram of FIG. 1, the removal method 100 comprises a sequence of consecutive steps for administering and applying a variety of constituents to effectively remove body tattoos, permanent make-up, and scalp micro-pigmentation. The removal method 100 overcomes the known disadvantages and problems associated with existing prior art in that it does not require the use of laser devices or technologies. Further, the initial application of the entire removal method 100 may take approximately 1.5 hours, or ninety minutes, to administer. This is significantly less than the time required for existing laser tattoo removal procedures. Depending on several factors (including the number of tattoos or target areas, the location of the tattoo(s), the skin depth and pressure the tattoo artist utilized in the creation of the tattoo, and the proper and consistent execution of applicable after-care instructions), a client may not require more than one initial application of the removal method 100 for full and permanent results.

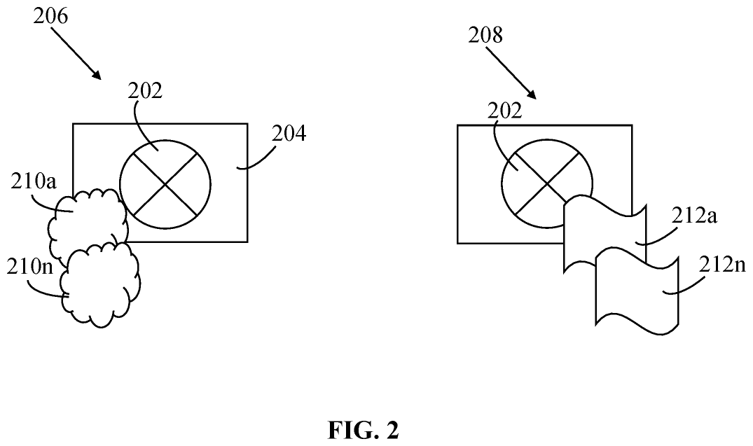

The removal method 100 begins with Steps 101 and 102, whereby Step 102 entails preparing a target area 202 where the body tattoo or permanent make-up selected for removal is disposed on the target area 202, located on a skin microbiome or epidermis 204 of the user. As best seen in FIG. 2, Step 102 comprises an initial act of cleaning 206 the target area 202. In preferred embodiments of the present invention, the initial act of cleaning 206 is carried out using widely available cleaning constituents such as bar soap, liquid soap, body wash, antibacterial gels, or disinfecting constituents. The purpose of Step 102 is to cleanse the epidermis 204 of bacteria, dirt, and other particles which might otherwise interfere with the effectiveness of the removal method 100 or infect the target area 202 once subsequent steps in the removal method 100 have been conducted. The cleaning 206 of the target area on the user's epidermis 204 may further be conducted using one or more absorbent materials 210a-n, wherein “n” represents any number greater than two, prior to the next steps in the removal method 100. In alternate embodiments, the at least one absorbent material 210a-n is of a textile material wherein “textile” is defined as any material, fabric, or cloth that is made from interlocking fibers, thin threads, filaments, or string yarn. For example, the at least one absorbent material 210a-n includes, without limitation, such materials as paper towels, towels, sponges, napkins, tissues, etc.

Step 102 further comprises a subsequent act of drying 208 the target area 202 after the initial act of cleaning 206 has been completed. In one exemplary embodiment, the act of drying 208 the target area 202 is conducted using one or more absorbent materials 212a-n that may be the same as or different than the materials 210a-n used to clean. Drying 208 ensures the target area 202 and epidermis 204 will be effectively receptive in absorbing the topical constituents and solutions utilized in subsequent steps of the removal method 100. A wet or damp epidermis 204 may interfere with or jeopardize the effectiveness of subsequent topical constituents and solutions utilized in the removal method 100.

In an alternate embodiment of the present invention and depending on the user's preference and/or pain tolerance, Step 102 may be followed by a Step 103 whereby a local anesthetic or other medication capable of temporarily numbing the target area 202 is applied to the target area 202 prior to Step 104. The purpose of Step 103 is to mitigate or altogether prevent the sensation or feeling of pain experienced by the user during Step 104. Step 103 is optional and may be skipped or replaced by a different pain management technique or practice depending, in part, on a user's preference.

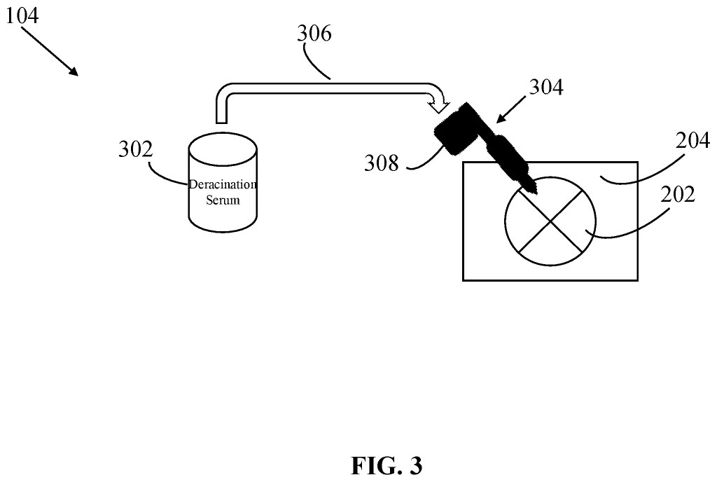

A further Step 104, as best depicted in FIG. 3, beneficially includes administering a deracination serum 302 to the target area 202 after cleaning 206 and drying 208 the target area 202 and, where applicable, after application of a local anesthetic to the target area 202 as set forth in Step 103. In a preferred embodiment of the present invention, the deracination serum 302 comprises the following ingredients: Water, Methylpropanediol, Bentonite, 1,2-Hexanediol, Aloe Barbadensis Leaf Extract, Kaolin, Propanediol, Butylene Glycol, Glycerin, Zinc Oxide, Silica, Sodium Chloride, Hydrolyzed Sodium Hyaluronate, Cynanchum Atratum Extract, Panthenol, Xylitol, Xylitylglucoside, Anhydroxylitol, Glucose, Hydroxypropyl Starch Phosphate, Polyacrylate Crosspolymer-6, Citric Acid, Ethylhexylglycerin, t-Butyl Alcohol, Disodium EDTA. Although the deracination serum 302 may also include a bentonite constituent, an aloe leaf extract constituent, and a hydrolyzed sodium hyaluronate constituent in preferred embodiments of the present invention, some or all these constituents may be removed or replaced in alternate embodiments.

By virtue of these ingredients and active properties, the function of the deracination serum 302 is to reach and loosen the pigmented material on the target area 202 without releasing the pigmented material into the user's blood stream. Rather than driving the extracted pigmented material deeper into the epidermis 204 or blood stream, like existing laser tattoo removal devices do, the deracination serum 302, in conjunction with proper use of the removal method 100, loosens the pigmented material and brings it to the outer surface of the epidermis 204, where it can be safely disposed of or wiped off in accordance with subsequent steps of the removal method 100.

In a preferred embodiment of the present invention, the deracination serum 302 is applied to the target area 202 with the use of a handheld automatic needle assembly having a plurality of needles 304 (hereinafter, “the needle assembly 304”) such as, for example, a magnetic coil or rotary tattoo machine or another comparable instrument, after the cleaning and drying of the target area 202. In those embodiments where a magnetic coil or rotary tattoo machine is used, the deracination serum 302 is first poured 306 into and stored in the tattoo ink reservoir 308. In alternate embodiments where other types of tattoo instruments 304 are utilized for completion of Step 104, the deracination serum 302 is poured 306 into the comparable tattoo ink reservoir 308 or other such feature of a substantially similar storage function. The deracination serum 302 is selectively administered onto the target area 202 using the needle assembly 304.

Depending on the type of the needle assembly 304 used in Step 104, the administering of the deracination serum 302 to the target area 202 may be facilitated or accomplished by puncturing the user's epidermis 204 with the plurality of needles on the needle assembly 304. In alternate embodiments, the needle assembly 304 used may not have the effect of puncturing the user's epidermis 204 and may achieve substantially the same goal and function, i.e., loosening and extracting the pigmented material, without the need for puncturing the user's epidermis 204.

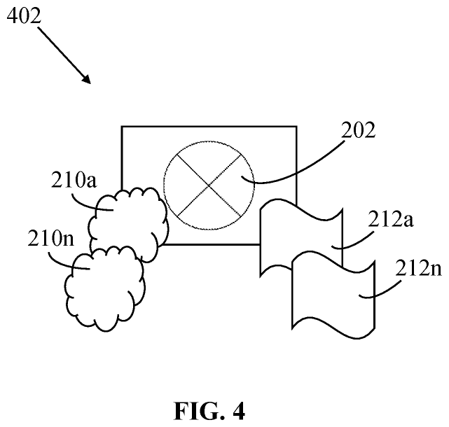

A further Step 106, as best depicted in FIG. 4, comprises wiping 402 the target area 202 clean with one or more absorbent materials 210a-n once Step 104 has been completed. Similar to the drying 208 of the target area 202 in Step 102, wiping 402 the target area 202 clean in this Step is conducted through the use of at least one absorbent or other materials 210a-n, 212a-n such as, by way of example, paper towels, towels, sponges, napkins, tissues, etc. Wiping 402 ensures the target area 202 and epidermis 204 will be effectively receptive in absorbing the topical constituents and solutions utilized in subsequent steps of the removal method 100. A wet or damp epidermis 204 may interfere with the effectiveness of subsequent topical constituents and solutions utilized in the removal method 100. In alternate embodiments, Step 106 may be optional, at the discretion of the user, but not required.

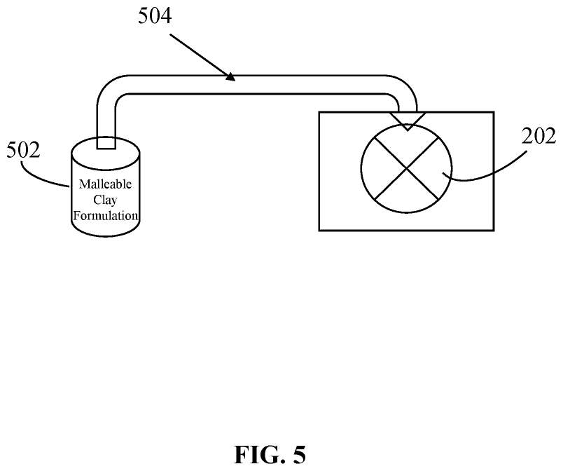

A next Step 108, as depicted in FIG. 5, comprises applying 504 a malleable clay formulation material 502, wherein “malleable” is defined as “easily influenced; pliable” and may include, by way of example, a cream, mask, or other malleable material, to cover the target area 202 and leaving the malleable clay formulation material 502 on the target area 202 until forming a dried-on clay formulation. In a preferred embodiment of the present invention, the malleable clay formulation material 502 comprises the following ingredients: Water, Methylpropanediol, Glycerin, 1,2-Hexanediol, Dipropylene glycol, Aloe Barbadensis Leaf Extract, Propanediol, Butylene glycol, Hydrolyzed Sodium Hyaluronate, Camellia Sinensis Leaf Extract, Oryza Sativa (Rice) Bran Extract, Sea Silt Extract, Tocopheryl Acetate, Lespedeza Capitata Leaf/Stem Extract, Ganoderma Lucidum (Mushroom) Extract, Cynanchum Atratum Extract, Trehalose, betaine, Allantoin, Arginine, Xanthan Gum, Glyceryl Acrylate/Acrylic Acid Copolymer, Acrylates/C10-30 Alkyl Acrylate Crosspolymer, PVM/MA Copolymer, Carbomer, PEG-40 Hydrogenated Castor Oil, Disodium EDTA, Ethylhexylglycerin, Caprylyl Glycol, Fragrance, Limonene, Linalool.

Although the malleable clay formulation material 502 is of a calcium bentonite clay material in preferred embodiments of the present invention, the calcium bentonite clay material may be removed or replaced in alternate embodiments of the invention.

By virtue of these ingredients and active properties, the function of the malleable clay formulation material 502 is to loosen and lift the pigmented material from the deeper levels of the epidermis 204 to the outer surface of the epidermis 204, where the extracted pigmented material can safely be disposed of or rinsed off in accordance with subsequent steps of the removal method 100. This feature of the removal method 100 overcomes some of the known disadvantages associated with the applicable prior art. Specifically, laser tattoo removal technologies drive the fragmented and loosened pigmented material deeper into the user's skin and possibly into the user's blood stream, thereby exposing the human body system to various ink residues and toxins. In contrast, the removal method 100 avoids this potentially harmful and dangerous result by virtue of the active ingredients and properties of the malleable clay formulation material 502 which lifts the extracted pigmented material rather than driving it deeper into the user's body.

The malleable clay formulation material 502 must be left on the target area 202 for a period of time sufficiently long enough for the malleable clay formulation material 502 to fully dry, cure, and form a dried-on clay formulation 604. Specifically, the dried-on clay formulation 604, once fully cured, is of a brittle property generally characterized by fracture upon subjection to stress, little deformation, poor capacity to resist impact and vibration of load, high compressive strength, and low tensile strength. In a preferred embodiment, the malleable clay formulation material 502 must be left on the target area 202 for a minimum of twenty (20) minutes for the dried-on clay formulation 604 to form. Additional time may be required depending on a variety of factors including, without limitation, the saturation of moisture or humidity in the air and in the user's epidermis 204. The mode of application of the malleable clay formulation material 502 onto the target area 202 varies in alternate embodiments of the present invention, varying from the utilization of wooden cosmetic spatulas to the user's fingers for more flexible distribution of the malleable clay formulation material 502.

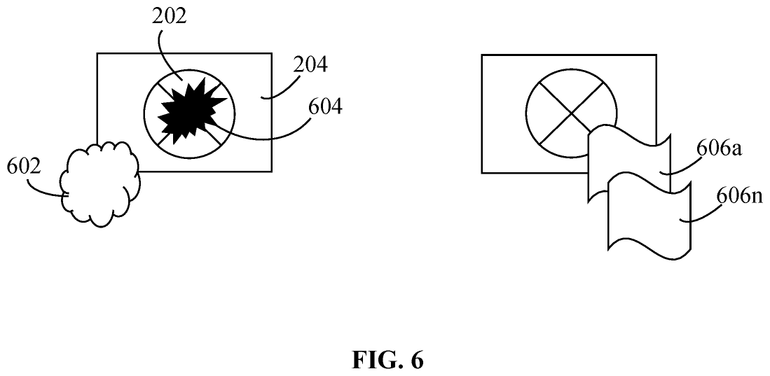

As best seen in FIG. 6, and after the dried-on clay formulation 604 has cured, a next Step 110 comprises removing the dried-on clay formulation 604 from the target area 202. In one embodiment, this may be accomplished by a preliminary act of cleaning 602 the target area 202 after Step 108. In preferred embodiments of the present invention, the initial act of cleaning 602 is carried out using widely available cleaning constituents such as bar soap, liquid soap, body wash, antibacterial gels, or disinfecting constituents. The chief function of Step 110 is to cleanse the epidermis 204 and remove the dried-on clay formulation 604 that the malleable clay formulation material 502 produced in Step 108. Further, Step 110 cleanses the epidermis 204 of bacteria, dirt, and other particles which might otherwise interfere with the healing of the target area 202 or infect the target area 202 if left thereon. The cleaning 602 of the target area 202 on the user's epidermis 204 may be conducted using the at least one absorbent material 210a-n (discussed above) prior to the next steps in the removal method 100. In alternate embodiments, the at least one absorbent material 210a-n is of a textile material wherein “textile” is defined as any material, fabric, or cloth that is made from interlocking fibers, thin threads, filaments, or string yarn. For example, the at least one absorbent material 210a-n includes, without limitation, such materials as paper towels, towels, sponges, napkins, tissues, etc.

Step 110 may, in some embodiments, further comprise a subsequent act of drying the target area 202 once the initial act of cleaning 602 has been completed. In one exemplary embodiment, the act of drying the target area 202 is conducted through the use of the at least one absorbent material 606a-n such as, for example, paper towels or cloth towels. Drying 606 ensures the target area 202 and epidermis 204 will be effectively receptive in absorbing the topical constituents and solutions utilized in subsequent steps of the removal method 100. Wet or damp epidermis 204 may interfere with the effectiveness of subsequent topical constituents and solutions utilized in the removal method 100.

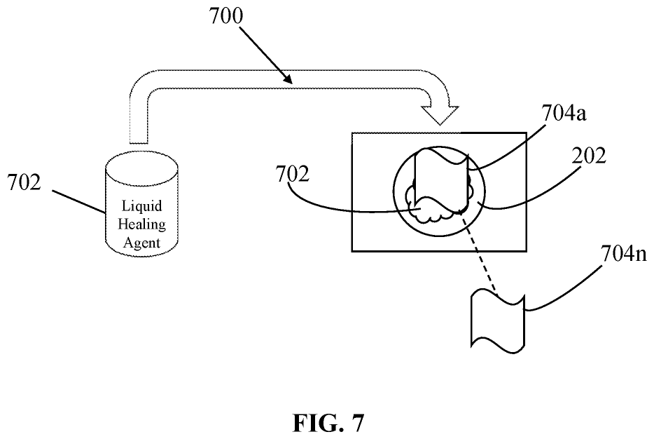

As depicted in FIG. 7, a final Step 112 comprises applying 700 a liquid healing agent 702 onto the target area 202 after removing the dried-on clay formulation 604 from the target area 202 and covering the liquid healing agent 702 with one or more transdermal drug delivery sheet(s) 704a-n, wherein “n” represents any number greater than two, for selective removal by the user after a healing time period. In a preferred embodiment of the present invention, the liquid healing agent 702 comprises the following ingredients: Water, Methylpropanediol, Glycerin, 1,2-Hexanediol, Dipropylene glycol, Aloe Barbadensis Leaf Extract, Leuconostoc/Radish Root Ferment Filtrate, Saccharomyces/Copper Ferment, Hydrolyzed Sodium Hyaluronate, Silver Sulfate, Panthenol, Arginine, Allantoin, Glucose, Anhydroxylitol, Butylene glycol, Xylitylglucoside, Xylitol, Propanediol, Ganoderma Lucidum (Mushroom) Extract, Trehalose, betaine, Cynanchum Atratum Extract, Carbomer, Caprylyl Glycol, Acrylates/C10-30 Alkyl Acrylate Crosspolymer, Glyceryl Acrylate/Acrylic Acid Copolymer, PVM/MA Copolymer, Xanthan Gum, PEG-40 Hydrogenated Castor Oil, Disodium EDTA, Ethylhexylglycerin, Fragrance, Limonene, Linalool. Although the liquid healing agent 702 is of a macromolecule healing constituent comprising a hyaluronic acid, peptide, and vitamin composition in preferred embodiments of the present invention, some or all these constituents may be removed or replaced in alternate embodiments of the present invention.

By virtue of these ingredients and active properties, the function of the liquid healing agent 702 is to heal the target area 202 upon application of all preceding Steps in the removal method 100. In a preferred embodiment of the present invention, the liquid healing agent 702 is applied on the target area 202 not only immediately following Step 110, but also at subsequent intervals of time, as needed to facilitate the natural healing process of the target area 202.

In a preferred embodiment, the healing time period, whereupon the liquid healing agent 702 and the at least one transdermal drug delivery sheet 704a-n remain on the target area 202 of the epidermis 204, ranges between approximately twenty-five (25) minutes and thirty (30) minutes.

In alternate embodiments, Step 112 may consist of first applying a liquid healing agent 702 to the target area 202 after removing the dried-on clay formulation 604 from the target area 202 and, after applying the liquid healing agent 702, covering the liquid healing agent 702 with the at least one transdermal drug delivery sheet 704a-n for selective removal by the user after a healing time period.

In one embodiment, Step 112 may be repeated several times, as needed and/or based on the user's preference, whereupon the liquid healing agent 702 is applied to the target area 202 after removing the dried-on clay formulation 604 from the target area 202 and the liquid healing agent 702 is covered with the at least one transdermal drug delivery sheet 704a-n for selective removal, which is then followed by replacement of another at least one transdermal drug delivery sheet 704a-n and liquid healing agent 702, by the user after the healing time period.

Step 112 may further comprise applying electrical stimulation to the target area 202 after the liquid healing agent 702 is applied to the target area 202 and the liquid healing agent 702 is covered with the at least one transdermal drug delivery sheet 704a-n. In one embodiment, this may be accomplished through iontophoresis treatments or another comparable form of transdermal drug delivery that utilizes electrical current to drive or push ionized drugs through the skin's outermost layer (stratum corneum). The removal method 100 concludes with Step 114.

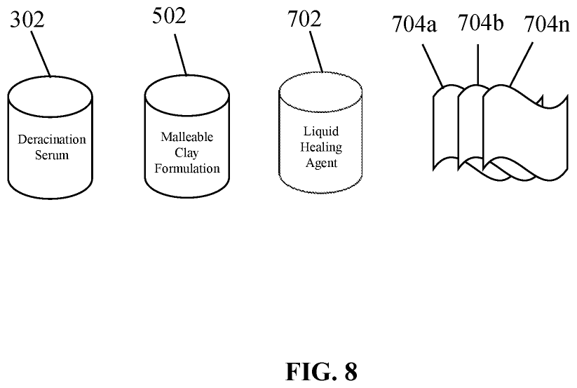

FIG. 8 depicts a schematic diagram of the products used in the present invention, wherein the deracination serum 302, the malleable clay formulation material 502, the liquid healing agent 702, and the at least one transdermal drug delivery sheet 704a-n are packaged separately to facilitate ease of use and storage of each such constituent. All constituents 302, 502, 702, and 704a-n are included in the removal method 100, though additional features, formulations, and topical constituents may accompany the removal method 100.

What is claimed is:

1. A non-laser tattoo removal method comprising the steps of:
cleaning and drying a target area on a user's skin, the target area including at least one tattoo disposed thereon;
administering a deracination serum to the target area with a handheld automatic needle assembly having a plurality of needles after the cleaning and drying of the target area;
wiping the target area with an absorbent material to remove an extracted pigmented material at least partially composing the tattoo;
applying a malleable clay formulation material to cover the target area and leaving the malleable clay formulation material on the target area until forming a dried-on clay formulation;
removing the dried-on clay formulation from the target area; and
applying a liquid healing agent to the target area after removing the dried-on clay formulation from the target area and covering the liquid healing agent with at least one transdermal drug delivery sheet for selective removal by the user after a healing time period.

2. The non-laser tattoo removal method according to claim 1, further comprising:
intermittently, during the administering of the deracination serum to the target area with the handheld automatic needle assembly, wiping the target area to remove an extracted pigmented material at least partially composing the tattoo.

3. The non-laser tattoo removal method according to claim 1, further comprising:
administering the deracination serum to the target area by puncturing the user's skin with the plurality of needles on the handheld automatic needle assembly.

4. The non-laser tattoo removal method according to claim 1, wherein:
the deracination serum includes a bentonite constituent, an aloe leaf extract constituent, and a hydrolyzed sodium hyaluronate constituent.

5. The non-laser tattoo removal method according to claim 1, wherein:
the malleable clay formulation material is of a calcium bentonite clay material.

6. The non-laser tattoo removal method according to claim 1, further comprising:
cleaning and drying the target area on the user's skin with at least one absorbent material before the administering of the deracination serum to the target area with the handheld automatic needle assembly.

7. The non-laser tattoo removal method according to claim 6, wherein:
the at least one absorbent material is of a textile material.

8. The non-laser tattoo removal method according to claim 1, further comprising:
cleaning and drying the target area on the user's skin with at least one absorbent material after removing the dried-on clay formulation from the target area.

9. The non-laser tattoo removal method according to claim 1, further comprising:
applying a local anesthetic to the target area before the administering of the deracination serum to the target area with the handheld automatic needle assembly.

10. The non-laser tattoo removal method according to claim 1, further comprising:
first applying a liquid healing agent to the target area after removing the dried-on clay formulation from the target area and, after applying the liquid healing agent, covering the liquid healing agent with at least one transdermal drug delivery sheet for selective removal by the user after a healing time period.

11. The non-laser tattoo removal method according to claim 1, further comprising:
applying the liquid healing agent to the target area after removing the dried-on clay formulation from the target area and covering the liquid healing agent with the transdermal drug delivery sheet for selective removal, and replacement of another transdermal drug delivery sheet and liquid healing agent, by the user after the healing time period.

12. The non-laser tattoo removal method according to claim 1, wherein:
the liquid healing agent is of a macromolecule healing constituent comprising a hyaluronic acid, peptide, and vitamin composition.

13. The non-laser tattoo removal method according to claim 1, further comprising:
applying electrical stimulation to the target area after the liquid healing agent is applied to the target area and the liquid healing agent is covered with the transdermal drug delivery sheet.
